(19) United States
(12) Patent Application Publication
(10) Pub. No.: US 2007/0185394 A1
(43) Pub. Date: Aug. 9, 2007

(54) Living body optical measurement apparatus

(76) Inventor: Takashi Ishizuka, Tokyo (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 11/572,084

(22) PCT Filed: Jun. 30, 2005
(86) PCT No.: PCT/JP05/12126

§ 371 Date: Jan. 12, 2007

(30) Foreign Application Priority Data
Jul. 15, 2004 (JP) 2004-208650

(51) Int. Cl. A61B 5/00; A61B 5/02

(52) U.S. Cl. 600/342; 600/504

(57) ABSTRACT

A measurement chair 7 is provided with a chair seat portion 8, a backrest portion 9, a pair of armrest portions 10, a pillow portion 11 provided at the upper portion of the backrest portion 9, a desk 12 provided on both armrest portions 10 and a jaw receiving portion which receives the jaw of the subject 1 and restricts a motion of the jaw. The pillow portion 11 receives the back head portion of the subject 1 and restricts a motion of the head of the subject 1. The desk 12 includes an inclined portion 12b arranged inclinedly with respect to a horizontal plane thereof. The jaw receiving portion 13 includes a telescope type column 13a of which base end portion is attached to a horizontal portion 12a of the desk 12 so as to permit free rotation in all directions and a contacting portion 13b which is provided at the top end portion of the column 13a for contacting the jaw. With such measurement chair of simple constitution, a motion of upper part of the subject is effectively restricted without giving the sense of oppression to the subject and a living body optical measurement apparatus, which permits a living body optical measurement with high measurement accuracy is obtained.

### FIELD OF THE INVENTION

The present invention relates to a living body optical measurement apparatus which measures a reaction information within a living body by making use of light at the time when a subject is seated on a measurement chair and is caused to carry out a task of drawing such as letters and pictures or a task relating to such as the sense of sight and the sense of hearing.

### BACKGROUND ART

There was a problem that during living body optical measurement, when a subject moves (body motion), such as an attachment position, angle or contact pressure of an optical fiber probe changes and a condition of optical passages change, thereby, the measurement accuracy reduces.

For this reason, conventionally, when a living body optical measurement is performed, while a subject is seated, for example, on a general-purpose chair and is caused to carry out a task relating to such as the sense of sight and the sense of hearing, the living body optical measurement was performed while securing the subject on the chair such as by means of a belt, however, with such measure since right and left motion of upper part of the subject can not be sufficiently restrained, thereby, a reduction of measurement accuracy was caused.

However, in order to sufficiently restrain such motion, when the subject is completely secured on the chair, the subject is placed under oppression, which disturbs a normal thinking power and a problem was caused which prevents a stable optical measurement.

In order to resolve such problems, for example, JP 5-154136 A discloses a living body optical measurement apparatus in which an existence and absence of a body motion of a subject is measured from a variation rate of signals such as detection signals and when an existence of body motion is measured, the signal level is returned to that of before the variation, thereby, the influence of a body motion is removed.

However, in such living body optical measurement apparatus, a possible body motion itself of a subject can not be prevented, thus, it was difficult to remove an influence of body motion.

An object of the present invention is to provide a living body optical measurement apparatus which uses a measurement chair which effectively secures proper limited portions of a subject with a simple constitutional member in a manner of reducing the sense of oppression given to the subject and effectively restricts a motion of upper part including a head portion of the subject, and improves the measurement accuracy as well as measurement efficiency.

### SUMMARY OF THE INVENTION

In a living body optical measurement apparatus according to the present invention, a measurement chair for seating a subject is provided with a simple constitutional member which suppresses the sense of oppression being given to the subject minimum and restrains the jaw or the chest of the subject and with the constitutional member, a motion of the upper part of the subject is effectively restricted, thereby, the measurement accuracy as well as the measurement efficiency in the living body optical measurement are improved.

When explaining the present invention further specifically, a living body optical measurement apparatus according to the present invention is provided with a measurement apparatus main body, a probe holder portion which is to be attached to a subject, a plurality of optical fibers which optically connect between the measurement apparatus main body and the probe holder portion and a measurement chair for seating the subject, wherein the measurement chair is provided with a jaw receiving portion which receives the jaw of the subject and restricts a motion of the jaw.

Further, a living body optical measurement apparatus according to the present invention is provided with a measurement apparatus main body, a probe holder portion which is to be attached to a subject, a plurality of optical fibers which optically connect between the measurement apparatus main body and the probe holder portion and a measurement chair for seating the subject, wherein the measurement chair is provided with a desk to be arranged in front of the subject seated on the measurement chair at the time of measurement and the desk is provided with an inclined portion arranged in an inclined manner with respect to the horizontal plane thereof.

Further, a living body optical measurement apparatus according to the present invention is provided with a measurement apparatus main body, a probe holder portion which is to be attached to a subject, a plurality of optical fibers which optically connect between the measurement apparatus main body and the probe holder portion and a measurement chair for seating the subject, wherein the measurement chair is provided with a pillow portion which receives the back of the head of the subject and restricts a motion of the head portion of the subject.

Further, a living body optical measurement apparatus according to the present invention is provided with a measurement apparatus main body, a probe holder portion which is to be attached to a subject, a plurality of optical fibers which optically connect between the measurement apparatus main body and the probe holder portion and a measurement chair for seating the subject, wherein the measurement chair is provided with a chest abutting member which is applied to the chest of the subject and restricts a motion of upper part of the subject.

Further, a living body optical measurement apparatus according to the present invention is provided with a measurement apparatus main body, a probe holder portion which is to be attached to a subject, a plurality of optical fibers which optically connect between the measurement apparatus main body and the probe holder portion and a measurement chair for seating the subject, wherein the measurement chair is provided with a light shielding hood for covering around the head portion of the subject.

In the living body optical measurement apparatus according to the present invention, since the measurement chair is provided with the jaw receiving portion which receives the jaw of the subject and restricts a motion of the jaw, a motion of the head portion of the subject can be prevented with such simple constitutional member without giving the sense of oppression to the subject, thus the measurement accuracy can be improved.

Further, since the measurement chair is provided with a desk to be arranged in front of the subject seated on the measurement chair at the time of measurement and the desk is provided with an inclined portion, for example, like a bookrack, arranged in an inclined manner with respect to the horizontal plane thereof, when the subject carries out a work such as drawing letters and pictures on the desk, a motion of the upper part can be limited and with such simple constitutional member the measurement accuracy can be enhanced.

Further, since the measurement chair is provided with the pillow portion which receives the back head portion of the subject and restricts a motion of the back head, a motion of the head portion of the subject can be prevented with such simple constitutional member without giving the sense of oppression to the subject, thus the measurement accuracy can be improved.

Further, since the measurement chair is provided with a chest member which is applied to the chest of the subject and restricts a motion of upper part of the subject, a motion of the upper part of the subject can be prevented with such simple constitutional member without giving the sense of oppression to the subject, thus the measurement accuracy can be improved.

Further, since the measurement chair is provided with a light shielding hood for covering around the head portion of the subject, influences due to external light from the circumference can be removed with such simple constitutional member, thus, the measurement accuracy can be improved.

### BRIEF DESCRIPTION OF THE DRAWINGS

FIG. 1 is a perspective view including in part a side view showing a living body optical measurement apparatus representing one embodiment according to the present invention.

FIGS. 2A and 2B are views for explaining the constitution of a pillow portion 11 of which position is adjustable in the present embodiment.

FIG. 3 is a view for explaining the structure of a bookrack type inclined portion 12b in the present embodiment.

FIG. 4 is a view for explaining the structure of a desk 12 of which position is adjustable in horizontal direction in the present embodiment.

FIGS. 5A and 5B are views for explaining respectively an inclination angle adjusting mechanism and a length adjusting mechanism of a telescope type column 13a in the present embodiment.

FIG. 6 is a perspective view showing a measurement chair in a living body optical measurement apparatus representing an embodiment 2 according to the present invention.

FIG. 7 is a perspective view showing a measurement chair in a living body optical measurement apparatus representing an embodiment 3 according to the present invention.

### BEST MODES FOR CARRYING OUT THE PRESENT INVENTION

Herein below, the best modes for carrying out the present invention will be explained with reference to the drawings.

### EMBODIMENT 1

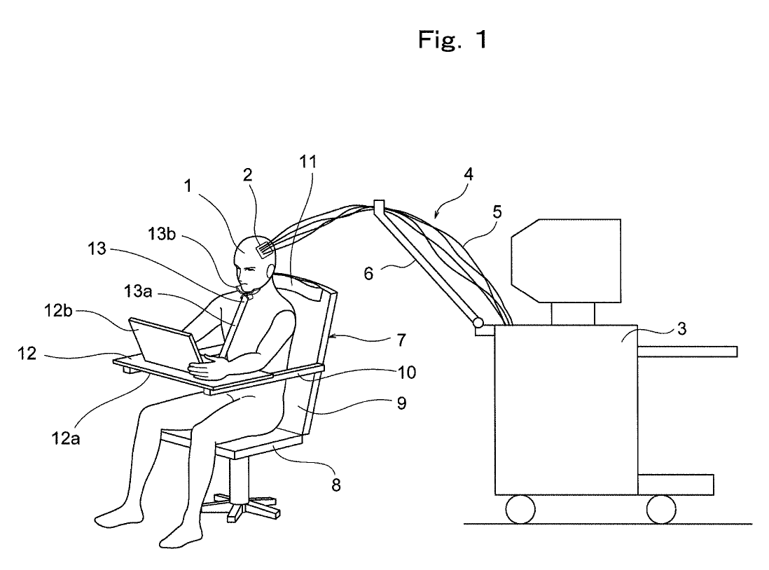

FIG. 1 is a perspective view including in part a side view showing a living body optical measurement apparatus representing one embodiment according to the present invention. In the drawing, at a head portion of a subject 1, a probe holder portion 2 is attached. The probe holder portion 2 is provided with a plurality (for example, 16 pieces) of optical measurement use probes (not shown) arranged in a grid shape.

A measurement apparatus main body 3 and the probe holder portion 2 are optically connected by a group of optical fibers 4. The group of optical fibers 4 contains a plurality (for example, 16 pieces) of optical fibers 5. A part of these optical fibers 5 are for irradiation use optical fibers for guiding detection light to the probes from the measurement apparatus main body 3, and the remaining optical fibers 5 are for light receiving use optical fibers for guiding the detection light reflected (scattered) within the subject 1 and transmitted therethrough to the measurement apparatus main body 3. A mid portion of the group of optical fibers 4 is supported by a top end portion of a supporting arm 6 extending from the measurement apparatus main body 3.

The living body optical measurement apparatus is provided with a measurement chair 7 for seating the subject 1 during measurement. The measurement chair 7 includes a chair seat portion 8, a backrest portion 9, a pair of armrest portions 10, a pillow portion 11 provided at an upper portion of the backrest portion 9, a desk 12 provided on both armrest portions 10 and a jaw receiving portion 13 which receives the jaw of the subject 1 and restricts a motion of the jaw.

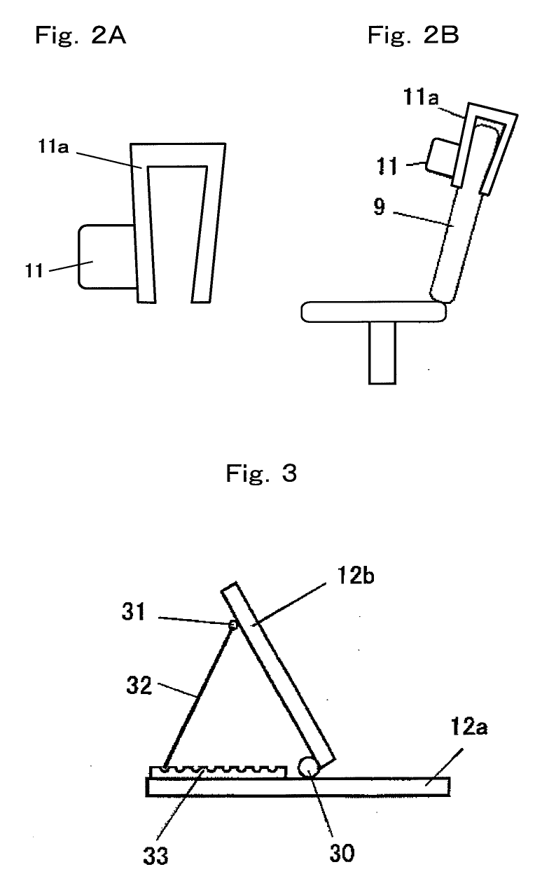

The pillow portion 11 receives the back head portion of the subject 1 and restricts a motion of the head of the subject 1. On the surface of the pillow portion 11, a curved depression for receiving the back head portion of the subject 1 is formed. As shown in FIGS. 2A and 2B, the pillow portion 11 is constituted in such a manner that the pillow portion 11 is detachable (exchangeable) with respect to the backrest portion 9 as well as the position in up and down direction of the pillow portion 11 is adjustable. Namely, the pillow portion 11 is secured to a pillow frame 11a, and the pillow frame 11a is inclined inside in a shape of leaf spring. Since the pillow portion 11 is retained any position on a face of the chair backrest 9 by catching the same by means of the spring reaction force of the pillow frame 11a, the position thereof can be easily adjusted according to the subject 1. Further, the pillow portion 11 is constituted by a sponge material so as to permit the thickness adjustment thereof. The right and left armrest portions 10 are disposed in parallel each other and horizontally.

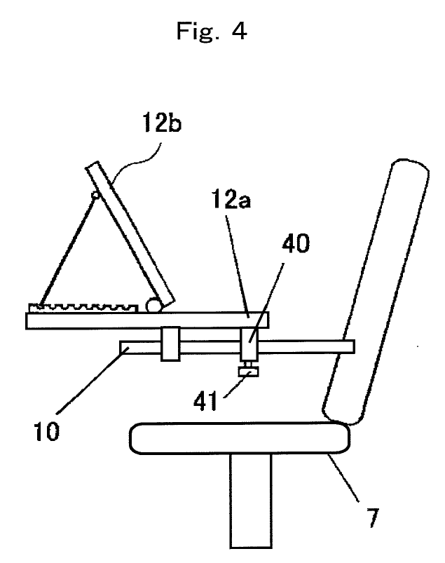

The desk 12 includes a horizontal portion 12a arranged horizontally on both armrest portion 10 and an inclined portion 12b, for example, like a bookrack arranged inclinedly with respect to the horizontal plane. As shown in FIG. 3, the inclined portion 12b is constituted so that the inclined portion is foldable on the horizontal portion 12a as well as the inclination angle thereof is adjustable by means of an inclined portion supporting member. Namely, the horizontal portion 12a and the inclined portion 12b are connected via a hinge 30, thereby, the inclined portion 12b is constituted to be foldable as well as the back face of the inclined portion is provided with a inclined portion leg 32 via a hinge 31 and grooves 33 are provided on the horizontal portion 12a, thus, when placing the inclined portion leg 32 in any one arbitrary groove, the angle of the inclined portion 12b is constituted to be adjustable. As shown in FIG. 4, the desk 12 is attached to one of the armrest portions 10 so as to permit rotation thereof using the same as an axis of rotation. When the subject 1 sits down on the measurement chair 7 or stands up therefrom, the space between the armrest portions 10 is opened by rotating the desk 12. Namely, when the subject 1 sits down on the chair, the desk is rotated around one of the armrests. Then, after the subject 1 sits down on the chair, the desk is returned to the original position and is arranged in front of the subject. Further, the same is true when the subject stands up from the chair. Further, the desk can be detachablely disposed with respect to the armrest portions 10. Further, in order that the distance from the subject 1 to the desk 12 is adjustable, the desk 12 is constituted so that the position thereof in horizontal direction is adjustable. Namely, there are provided with a horizontal column constituting the armrest portion 10 attached to the measurement chair 7, holder 40 passed through the horizontal column, the desk 12 attached to the holder 40 and a fastening screw 41 provided for the holder 40, when the fastening screw 41 is loosened, the holder 40 passed through the horizontal column is rendered movable and the distance between the desk 12 and the chair 7 can be adjustable. Through fastening the fastening screw 41 the desk is secured immovable.

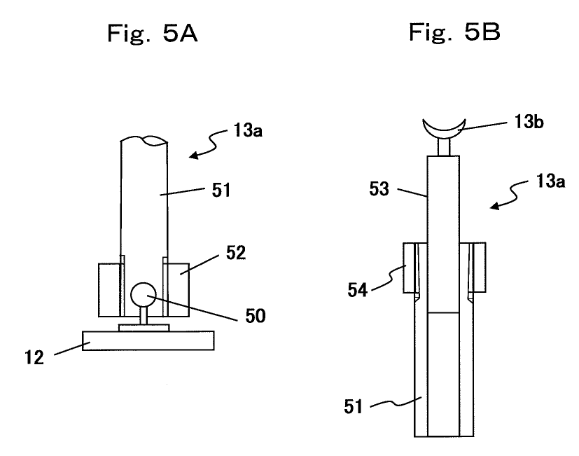

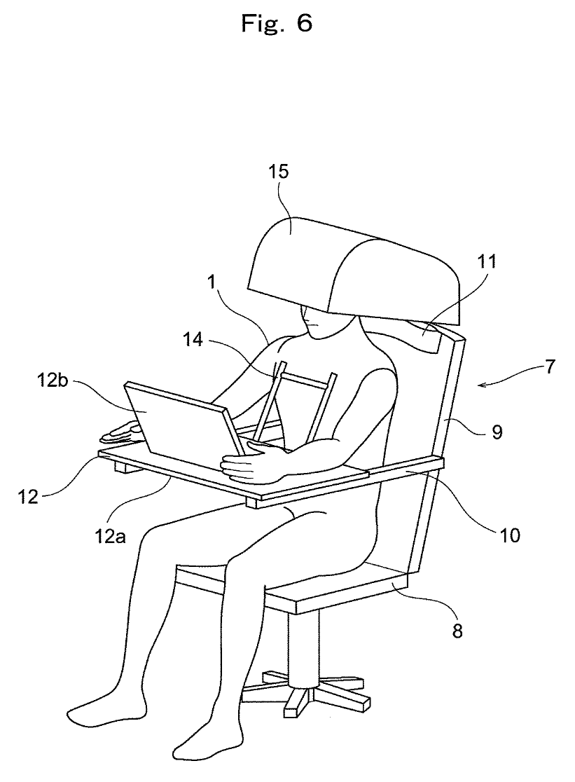

The jaw receiving portion 13 includes a telescope type column 13a of which base end portion is connected to the horizontal portion 12a of the desk, for example, as shown in FIG. 5A via a ball bearing permitting rotation in all directions and a nearly U shaped contacting portion 13b which is provided at the top end portion of the column 13a and is to be contacted to the jaw. Further, through fastening a fastening screw provided at the column 13a, the inclination angle of the column 13a is kept at any angle. Namely, the inclination angle adjustment mechanism is constituted by a ball shape column 50 of which top end is formed in a ball shape, an outer column 51 at which bottom end outer circumference a groove for a male screw and at which bottom end a ball shaped aperture for receiving the ball shaped portion of the ball shaped column 50 are provided and a nut 52 for a taper screw, the ball shaped column 50 is secured to the desk 12, the nut 52 is fitted to the male screw of the outer column and the ball shaped portion of the ball shaped column 50 is pushed in into the outer column 51. Through fastening the nut 52, the portion of the outer column 51 provided with the groove is narrowed, thereby, the outer column 51 and the ball shaped column 50 is fixed. When loosening the nut 52, the portion of the outer column 51 provided with the groove is expanded, the fixing is released to permit the angle adjustment of the outer column 51 with reference to the ball shaped column 50 as a fulcrum. Further, the telescope type column 13a is constituted as shown in FIG. 5B so that the length thereof is adjustable by fastening a fastening screw provided at a mid portion thereof. Namely the length adjustment mechanism is constituted by an inner column 53 provided with at the top thereof the jaw receiving portion 13b, the outer column 51 of a hollow structure at which top end outer circumference a groove for a male screw is provided and a nut 54 for a taper screw, when the nut 54 is fitted to the male screw of the outer column 51, the inner column 53 is inserted into the hollow outer column 51 and the nut 54 is fastened, the portion of the outer column 51 provided with the screw groove is narrowed and thereby, the outer column 51 and the inner column 53 are fixed. When the nut 54 is loosened, the portion of the outer column 51 provided with the groove is expanded, and the fixing is released to permit the length adjustment.

In such living body optical measurement apparatus, since the jaw receiving portion 13 for restricting a motion of the jaw of the subject 1 is provided for the measurement chair 7 via the desk 12, a motion of the head portion of the subject 1 can be prevented with such simple constitutional member without giving the sense of oppression to the subject 1, thus the measurement accuracy can be improved. In particular, during measurement when the subject 1 is caused to draw such as letters and pictures, a motion of the head portion of the subject 1 can be prevented, thus, the measurement accuracy as well as the measurement efficiency can be improved.

Further, since the inclination angle and the length of the column 13a is adjustable, and the jaw can be received at an optimum position according to the body size of the subject 1, a possible load imparted to the subject 1, namely a task execution and measurement under an unnatural posture is minimized.

Further, since the measurement chair 7 is provided with a desk 12 to be arranged in front of the subject 1 seated on the measurement chair 7 at the time of measurement and the desk 12 is provided with an inclined portion 12b arranged in an inclined manner with respect to the horizontal plane thereof, when the subject 1 is caused to carry out a work such as drawing letters and pictures on the desk, a motion of the upper part of the subject 1 can be limited, thereby, with such simple constitutional member the measurement accuracy can be improved. Further when comparing with a case where a desk separate from the chair is used, the transportation and storage of the desk 12 are easy as well as an optimum environment for a measurement can be quickly prepared.

Further, since the inclination angle of the inclined portion 12b can be adjustable, and the inclination angle of the inclined portion 12b can be adjusted at an optimum angle according to the body size of the subject 1, a motion of the upper part of the subject 1 can be further limited.

Further, since the distance from the subject 1 to the desk 12 is adjustable, the desk 12 can be disposed at a position where the subject 1 can carry out a task easily according to the body size of the subject 1, and from this point of view, a possible load imparted to the subject 1 is minimized.

Still further, in the present embodiment, since the combination of the jaw receiving portion 13 and the inclined portion 12b of the desk is used, the measurement accuracy, in particular, when the measurement is performed while causing the subject 1 to draw such as letters and pictures is further effectively improved.

Further, since the measurement chair 7 is provided with the pillow portion 11 which receives the back head portion of the subject 1 and restricts a motion of the back head, when the subject 1 carries out a task relating to such as the sense of sight and the sense of hearing under a condition resting on the backrest portion 9, a motion of the head portion of the subject 1 can be prevented with such simple constitutional member without giving the sense of oppression to the subject 1, thus, the measurement accuracy can be improved. Further, the desk is not necessarily provided for the chair. For example, when a plurality of legs is provided for the desk, the desk can be disposed independently from the chair.

### EMBODIMENT 2

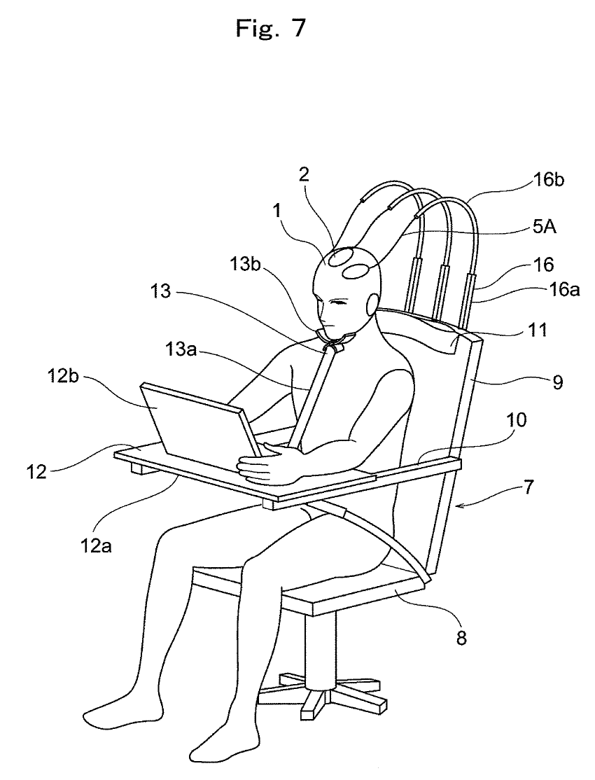

Now, FIG. 6 is a perspective view showing a measurement chair in a living body optical measurement apparatus representing an embodiment 2 according to the present invention. In the drawing, the measurement chair 7 is provided via the desk 12 with a chest-abutting member 14, which is applied to the chest of the subject 1 and restricts a motion of upper part of the subject 1. The base end portion of the chest-abutting member 14 is swingablly connected to the horizontal portion 12a of the desk. By fastening a fastening member (not shown) provided for the chest abutting member 14, the inclination angle of the chest abutting member 14 can be kept at any angle.

Further, the measurement chair 7 is provided with a light shielding hood 15 for covering around the head portion of the subject 1. The light shielding hood 15 is rotatablly attached to the top end portion of the backrest portion 9. The light shielding hood 15 is made from a light shielding material such as a light shielding cloth and shields external light irradiated to the probe holder portion 2. The constitutions other than those explained above are the same as those in embodiment 1.

In such living body optical measurement apparatus, since the measurement chair 7 is provided with via a desk a chest-abutting member 14 which is applied to the chest of the subject 1 and restricts a motion of the upper part of the subject 1, a motion of the upper part of the subject 1 can be prevented with such simple constitutional member without giving the sense of oppression to the subject 1, thus, the measurement accuracy can be improved.

Further, since the measurement chair 7 is provided with a light shielding hood 15 for covering around the head portion of the subject 1, influences due to external light from the circumference can be removed with such simple constitutional member, thus, the measurement accuracy can be improved.

Embodiment 3 Now, FIG. 7 is a perspective view showing a measurement chair in a living body optical measurement apparatus representing an embodiment 3 according to the present invention. Although to each of the three probe holder portions 2 a plurality of optical fibers 5 are respectively connected as in the same manner as in FIG. 1 embodiment, optical fibers 5 which are connected to a same probe holder portion are respectively collected within one of cables 5A, 5B and 5C. The measurement chair 7 is provided with corresponding three fiber guides 16, which respectively hang down the cables 5A, 5B and 5C at the end portions thereof near the probe holder portions 2 as well as guide the respective cables 5A, 5B and 5C near to the head portion of the subject 1.

Each of the fiber guides 16 includes a guide main body 16a, which is secured to the backrest portion while protruding from the top end portion of the backrest portion 9 and a curved portion 16b led out from the guide main body 16a. The curved portion 16b is constituted by an elastic deformable material in a tube shape and the top portion thereof is curved so as to face the head portion of the subject 1. The respective cables 5A, 5B and 5C are inserted in the corresponding fiber guides 16 and are led out from the top end portions of the curved portions 16b.

The chair seat portion 8 is provided with a fixing use belt 17 for fixing the waist portion of the subject 1 to the measurement chair 7. Other constitutions than those explained above are the same as those explained in embodiment 1.

In the above living body optical measurement apparatus, since the measurement chair 7 is provided with the fiber guides 16, cables 5A, 5B and 5C are prevented from intertwining as well as an efficiency of attaching work of the probe holder portions to the subject 1 is improved. Further, since the optical fibers 5 are respectively collected in groups and their movements are suppressed, the measurement accuracy is improved.

Still further, since the measurement chair 7 is provided with the fixing use belt 17 and a motion of the subject 1 can be restricted, the measurement accuracy is improved.

1. A living body optical measurement apparatus comprising: a measurement apparatus main body, a probe holder portion which is to be attached to a head portion of a subject for measuring blood flow in the brain, optical fibers one ends of which are connected to the measurement apparatus main body and the other ends of which are connected to the holder portion for optically connecting both, a measurement chair for seating the subject and a desk which is arranged facing the measurement chair and serves the subject seated on the measurement chair during measurement for performing a work for the measurement, wherein on the horizontal face of the desk a fixing member for supporting a predetermined portion of the subject is provided.

2. A living body optical measurement apparatus according to claim 1, wherein the measurement chair includes a seating portion and a back face portion for supporting the back face of the subject and the desk includes a pair of arm supporting portions extending from the end faces of the back face portion toward the subject and a flat face portion arranged horizontally and extending in a direction perpendicular to the pair of the supporting portion, and on the flat face portion the fixing member for supporting a predetermined portion of the subject is provided.

3. A living body optical measurement apparatus according to claim 1 or 2, wherein the desk is provided with an inclined portion arranged inclinedly with respect to the horizontal face of the desk.

4. A living body optical measurement apparatus according to claim 1, wherein the fixing member for the subject is a jaw receiving member which receives the jaw of the subject and restricts a motion of the jaw.

5. A living body optical measurement apparatus according to claim 4, wherein the jaw receiving member is a telescope type member which is free to rotate in all directions.

6. A living body optical measurement apparatus according to claim 1, wherein the fixing member for the subject is a chest abutting member which is applied to the chest portion of the subject and restricts a motion of the upper part of the subject.

7. A living body optical measurement apparatus according to claim 2, wherein the back face portion is provided with a pillow portion which receives the back head portion of the subject and fixes the head portion of the subject.

8. A living body optical measurement apparatus according to claim 2, wherein the back face portion is provided with a light shielding hood which covers around the head portion of the subject.

9. A living body optical measurement apparatus according to claim 2, wherein the back face portion is provided with a fiber guide which hangs down the end portions of the optical fibers at the side of the probe holder as well as guides the optical fibers near to the head portion of the subject.

10. A living body optical measurement apparatus according to claim 1, wherein the measurement chair is provided with a fixing use belt for fixing the waist portion of the subject.

11. A living body optical measurement apparatus according to claim 2, wherein the flat face portion is provided with an inclined member arranged inclinedly with respect to the horizontal face of the desk and the inclined member is provided with a mechanism which permits to vary freely the inclination angle of the inclined member.

12. A living body optical measurement apparatus according to claim 2, wherein the flat face portion is permitted to slide horizontally.

13. A living body optical measurement apparatus according to claim 2, wherein the flat face portion is permitted to rotate around the center axis of one of the arm supporting portion.

14. A living body optical measurement apparatus according to claim 4, wherein the jaw receiving portion is permitted to expand and to contract.
